(19) United States
(12) Patent Application Publication
(10) Pub. No.: US 2006/0067906 A1
(43) Pub. Date: Mar. 30, 2006

(54) Extended wear low viscosity cosmetic compositions

(76) Inventor: Angie Sanders, Lewisburg, TN (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/211,817

(22) Filed: Aug. 25, 2005

Related U.S. Application Data
(60) Provisional application No. 60/613,138, filed on Sep. 24, 2004.

(51) Int. Cl. A61K 8/81; A61K 8/89

(52) U.S. Cl. 424/70.12; 424/70.16

(57) ABSTRACT

Extended wear compositions are disclosed which include the use of silicone resin, a dimethicone solvent, a low viscosity dimethicone dispersant having a viscosity of less than 1,000 cps and a high concentration of colorant or pigments. The resultant low viscosity compositions can be dispensed with a felt tip applicator and still provide extended wear qualities.

### CROSS REFERENCE TO RELATED APPLICATION

The benefit under 35 U.S.C. §119(e) of U.S. Provisional Patent Application Ser. No. 60/613,138 filed Sep. 24, 2004, is hereby claimed.

### BACKGROUND

1. Technical Field

Improved extended wear and low viscosity cosmetic compositions are disclosed that are suitable for use as lip liners, eyeliners, eye shadows, blushes, concealers and other cosmetic preparations where an extended wear quality is desired.

2. Brief Description of Related Technology

Extended wear cosmetics are in substantial demand. Specifically, today's busy consumer has little time to reapply cosmetic preparations during the workday and substantial amounts of time are expended in applying cosmetics at the beginning of the day. Because many consumers apply the same type of cosmetic preparation day after day, a demand has arisen for permanent cosmetics in the form of eyeliner tattoos so that eyeliner does not have to be reapplied or ever again.

One problem associated with permanent eyeliner or eyeliner tattoos is that the fashion of cosmetics changes over time and a consumer with a permanent eyeliner tattoo may prefer a different look in the future. Therefore, permanent cosmetics such as eyeliner tattoos are clearly not preferable due to the continuous evolution and changing of styles, tastes, and trends in cosmetics.

Therefore, to save time, consumers have often turned to extended wear cosmetics. Some extended wear cosmetics preparations are intended to last anywhere from six to twelve hours. Such extended wear cosmetics preparations are typically solid or semi-solid formulations. They often contain oils, waxes, esters and other common raw materials to help thicken and solidify the extended wear formulas. By using such thickeners in large amounts, the extremely viscous extended wear formulations are not prone to settling and separation. However, if such separation and settling does occur, the product normally cannot be successfully re-mixed by the consumer.

With the use of such thickeners and structure building materials, the amount of colorant and film formers are reduced, which is disadvantageous. Thus, by constraining the amount of colorant and film former, the product performance is compromised.

Therefore, there is a need for extended wear cosmetics that are less reliant upon thickeners and structure builders such as waxes, esters, gums and other common cosmetic raw material thickeners and which contemporaneously include increased amounts of colorants and film formers so that they are more effective than conventional eyeliner and lip liner compositions.

### SUMMARY OF THE DISCLOSURE

An extended wear cosmetic composition that is suitable for use as an eyeliner, lip liner, concealer, blush, eye shadow and other uses is disclosed. The disclosed composition comprises a silicone resin, a volatile dimethicone solvent and a color paste that includes one or more low viscosity dimethicones. The dimethicone preparation of the color paste functions as a colorant dispersant due to its low viscosity, that is preferably less than 1,000 cps. The volatile dimethicone that is used as a solvent has a viscosity of around 1 cps or less.

In a refinement, the dimethicone(s) used in the color paste has a viscosity ranging from about 5 cps to about 900 cps. In a further refinement, the color-paste dimethicone(s) has a viscosity of about 30 cps to about 450 cps. In still a further refinement, the color-paste dimethicone(s) has a viscosity of about 120 cps to about 300 cps.

In another refinement, the composition has a viscosity in a range of about 10 to 1000 cps.

In still another refinement, the composition comprises at least one film-forming agent. In a further refinement of this concept, the film-forming agent comprises a hydrophobic acrylic copolymer. In still a further refinement, the hydrophobic acrylic copolymer is carboxylated and has a molecular weight in excess of 10,000 daltons (Da).

In a further refinement, the film-forming agent is supplied in a solvent, for example isopropyl alcohol.

In another refinement, the silicone resin comprises trimethylsiloxysilicate.

In another refinement, the volatile dimethicone solvent comprises hexamethyldisiloxane.

In another refinement, the colorant comprises isopropyl titanium triisostearate (ITT).

In a further refinement, the silicone resin is present in an amount ranging from about 10% by weight, based on the weight of the cosmetic composition (wt. %) to 30 wt. %. The dimethicone solvent is present in an amount ranging from about 35 wt. % to 55 wt. % and more than 20 wt. % of the formulation consists of the colorants in the form of color pastes.

In a further refinement, the color paste comprises about 60 wt. % silicone resin and dimethicones having a viscosity of less than 1,000 cps, and about 40 wt. % colorant. Thus, the color paste may comprise a combination of silicone resin, low viscosity dimethicones and colorant. The low viscosity dimethicones may be polydimethylsiloxanes in the form of AK 35™, AK 350™ and combinations (e.g., mixtures) thereof sold by Wacker Chemical Corporation.

In another refinement, the film-forming agent is present in an amount ranging from about 0.25 to about 1 wt. %.

A method for preparing an extended cosmetic composition is disclosed. The method comprises mixing a silicone resin with a dimethicone solvent blending one or more colorants with additional silicone resin and one or more low viscosity dimethicones having a viscosity of less than 1,000 cps to form a colorant paste and adding the paste to the mixed silicone resin and dimethicone; optionally adding a film-forming agent, at least one resin selected from the group consisting of silica resin, polytetrafluroethylene resin, and mixtures thereof, and at least one preservative, and mixing the resultant product.

### BRIEF DESCRIPTION OF THE DRAWINGS

An applicator for applying the disclosed eyeliners and lip liners is described more or less diagrammatically in the accompanying drawing, wherein:

FIG. 1 is a side sectional view illustrating an applicator assembly for dispensing the disclosed eyeliners and lip liners;

FIG. 2 is a side sectional view illustrating the applicator assembly of FIG. 1 with the cap removed;

FIG. 3 is a side sectional view of the applicator cap;

FIG. 4 is a side plan view of the tip or nib of the applicator shown in FIGS. 1-3;

FIG. 5 is a side sectional view of the tip or nib holder of the applicator shown in FIGS. 1-4;

FIG. 6 is a side sectional view of the mixing bar accommodated partially within the nib holder of FIG. 5 and partially within the cartridge casing of FIG. 8;

FIG. 7 is a sectional view of the mixing ball disposed between the mixing bar of FIG. 6 and the cartridge casing of FIG. 8;

FIG. 8 is a side sectional view of the cartridge casing partially disposed within the barrel of FIG. 9 and which partially accommodates the nib holder of FIG. 5 and a mixing bar of FIG. 6 and which accommodates the mixing ball of FIG. 7; and

FIG. 9 is a side sectional view of the barrel of the applicator as shown in FIGS. 1 and 2.

### DETAILED DESCRIPTION OF EMBODIMENTS

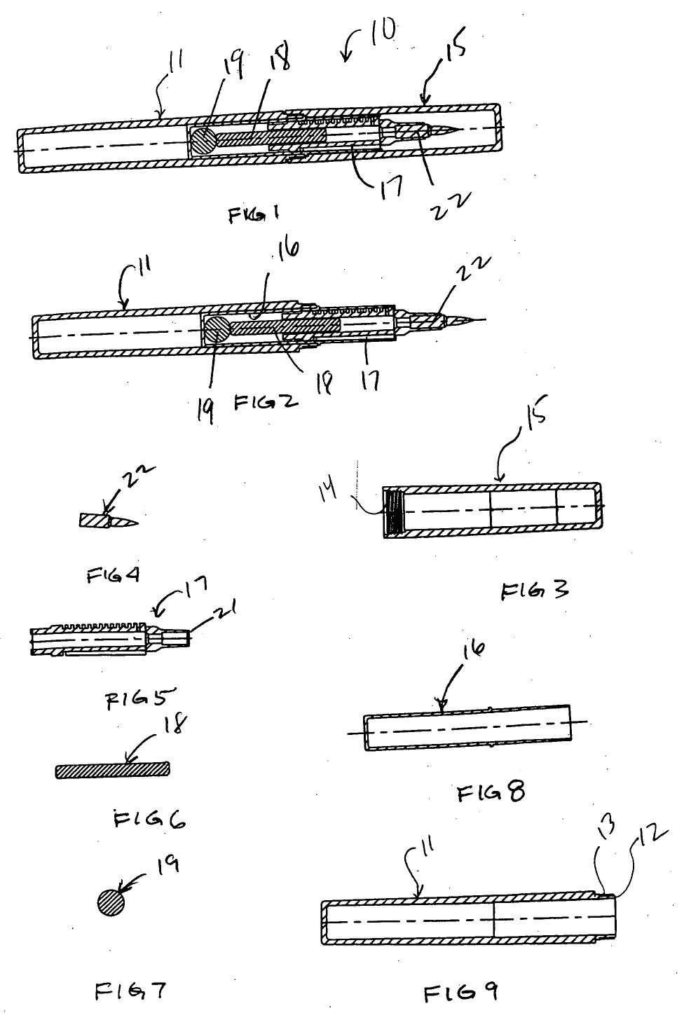

Disclosed herein are relatively low viscosity compositions that can function as extended wear eyeliner and lip liner formulations. The disclosed compositions have a viscosity that enables them to be delivered by way of a felt tip applicator, such as that shown in FIGS. 1-9. The combination of low viscosity dimethicones, silicone resins, and high concentrations of colorants enable the low viscosity formulations disclosed herein to embody extended wear properties even though they can be delivered through a felt tip applicator or similar applicator.

While extended wear cosmetic applications have been available, no such extended wear cosmetic applications are believed to be substantially free of waxes, esters, oils, gums and other thickening agents or dimethicone preparations with viscosities exceeding 1,000 cps. Accordingly, in one type of embodiment, the compositions described herein are comprehended to be free of or substantially free of waxes, esters, oils, gums, other thickening agents, and dimethicone preparations with viscosities exceeding 1,000 cps. In addition to a volatile dimethicone solvent, the disclosed formulations can utilize low viscosity dimethicones in the color paste mixture, for example those with viscosities of less than 1,000 cps and preferably less than 500 cps.

Overall, the cosmetic composition preferably has a viscosity of 1000 cps or less, 700 cps or less, and more preferably 500 cps or less. The composition preferably has a viscosity of at least 10 cps, or at least 25 cps, and more preferably at least 40 cps. Preferred ranges include about 10 cps to about 1000 cps, about 25 cps to about 700 cps, and about 40 cps to about 500 cps.

Hexamethyldisiloxane and polydimethylsiloxane are dimethicones that have been previously used in antiperspirant and deodorant delivery systems, cuticle coating products and high viscosity color cosmetic compositions. Dimethicones like polydimethylsiloxane are color dispersants which help to uniformly spread pigments over the surface of skin and hair. They provide enough time commonly referred to in the cosmetic's industry as “playtime” to supply sufficient rub-in properties, and reduce tackiness. Dimethicones can also impart lubricity and a smooth skin feel. Previously, dimethicones that have been used in the cosmetics industry are provided in the form of a high viscosity fluid or gum. Specifically, U.S. Pat. No. 6,406,683 (Jun. 18, 2002) teaches the use of dimethicone gums having viscosities of greater than 2.5 million cps. The '683 patent also teaches the mixture of dimethicones with organosiloxane resins, but the dimethicones have viscosities exceeding 1,000 cps.

In contrast, the disclosed formulations utilize dimethicones as a solvent and as a dispersant/solvent in color pastes. The dimethicones in the color pastes have a low viscosity, of less 1,000 cps, preferably less than 500 cps and more preferably in the range of 30 cps to 450 cps, most preferably 120 cps to 300 cps. In another embodiment, the dimethicones in the color pastes have a viscosity in a range of 250 to 450 cps, e.g. about 350 cps. The volatile dimethicone solvent has a negligible viscosity, around 1 cps or less.

The dimethicone solvent is combined with a silicone resin. A preferred silicone resin is trimethylsiloxysilicate (TMS). TMS is an excellent film former and is suitable for use in cosmetic applications. Previously, TMS has been combined with high viscosity-dimethicones to produce thickened extended wear products. However, disclosed herein are low viscosity compositions for use as eyeliners; and lip liners and that can be made to be dispensed with felt tip applicators. Preferred dimethicone solvents are linear siloxanes, such as hexamethyldisiloxane. One suitable source of hexamethyldisiloxane is Wacker Chemical Corporation of Munich, Germany and its WACKER-BELSIL DM 0.65 brand of hexamethyldisiloxane. WACKER-BESLSIL DM 1 plus, also sold by Wacker Chemical Corporation, may be used. While other volatile hydrocarbons can be used as the solvent, their evaporation rates all tend to be drastically different from disiloxane and therefore change the overall wear characteristics of the overall product. These volatile hydrocarbons conform to having from about C₈ to about C₂₀ chain lengths or have 3, 4, and 5 membered ring structures. Said volatiles could include such isoparraffins as isododecane, isohexadecane, isococosane, and the like. Volatile cyclomethicones could include 244 fluid, 245 fluid, 344 fluid, and 345 fluid all from Dow Corning Corporation.

The combination of the silicone resin and the dimethicone can make up to about 75 wt. % of the total composition, preferably about up to 70 wt. %. The silicone resin can be present in an amount from about 10 wt. % to 30 wt. %, more preferably from about 17 wt. % to about 25 wt. % with preferred values between 20 wt. % and 25 wt. %. At these levels, e.g., 20 wt. % to 25 wt. %, the low viscosity dimethicone can be present in an amount ranging from about 30 wt. % to about 60 wt. %, more preferably from about 40 wt. % to about 50 wt. %, more preferably from 44 wt. % to about 49 wt. %.

With the accumulated amounts of silicone resin and dimethicones hovering at or below 75 wt. % and most preferably slightly below 70 wt. %, large quantities of colorants in the form of color pastes, such as amounts approaching 30 wt. % color paste may be utilized.

In a preferred method, after mixing the silicone resin and the dimethicone solvent with a propeller mixer or other suitable mixer until a clear homogeneous mixture is provided, the colorant is then added. The colorant preferably is provided in the form of a color paste. The color paste is made by known cosmetic procedures for grinding pigments. For example, preferably the colorants are ground in a dimethicone and silicone resin blend by passing the mixture through a roller mill until desired grind is created. The color paste makes up the bulk of the remaining portion of the composition with other, smaller amounts attributable to film-forming additives, silica, polytetrafluoroethylene, and preservatives. Thus, if the silicone resin and dimethicone account for 68-69 wt. % of the composition, up to about 30 wt. % may be used for the color paste, for example.

In a preferred embodiment, the color paste comprises a combination of pigment, a pigment treatment such as isopropyl titanium triisostearate, one or more silicone resins and dimethicones with a viscosity of less than 1,000 cps, preferably less than 500 cps, and more preferably from 250 to 450 cps. Preferred dimethicones for the color pastes are AK 35™ and AK 350™, both polydimethylsiloxanes, and both sold by Wacker Chemical Corporation. They have viscosities of 33.1 cps and 232 cps, respectively. A mixture of TMS resin and polydimethylsiloxanes can be used which preferably would have a viscosity of about 300 cps, as the powdered TMS resin increases the viscosity of the polydimethylsiloxane mixture (e.g., AK 353 and AK 3503).

Various pigment treatments are available, such as surface treatments. Preferred treated pigment are not hydrophilic, and preferably are hydrophobic or both hydrophobic and lipophilic, such as through a perfluoro treatment. Isopropyl titanium triisostearate-treated pigments are preferred. Another treatment includes ITT and silane (e.g., ITT/triethyoxycaprylylsilate), ITT and methicone or ITT and dimethicone, to form a crosspolymer at the surface of the pigment, making them dispersible in esters, silicones and hydrocarbons. Other treatments contemplated include branched alkyl silane treatment, amino silicone treatment, silane treatment, and hybrid treatments (e.g., perfluoroalcohol phosphate and methicone, acryl silicone copolymer or silane in one step, without forming a crosspolymer). Suitable ITT-treated pigments are available from Kobo Products, Inc., of South Plainfield, New Jersey. See also U.S. Pat. No. 4,877,604, the disclosure of which is hereby incorporated herein.

The ratio of colorant (pigment, die, or combination thereof) and isopropyl titanium triisostearate to silicone resin ranges from about 1:1 to about 6:1. The color paste also preferably includes one or more low viscosity dimethicones to improve the dispersability of the colorant. The ratio of colorant (pigment, die, or combination thereof) and isopropyl titanium triisostearate to dimethicones is about 1:1 to about 1:4. A pearlescent (pearl) colorant can also be used, in approximately the same ratios to silicone resin and dimethicones, and is preferably added in a separate step rather than ground in with the other colorant(s). It is believed that use of a pearl colorants in presently-available commercial forms will not allow their dispensation through a flow-through pen, although they will have benefit in other applicators.

A secondary film-forming agent may be added, and preferably includes a hydrophobic acrylate copolymer, more preferably a carboxylated hydrophobic acrylic copolymer and still more preferably a carboxylated hydrophobic acrylic having a high molecular weight, preferably in excess of 10,000 Da. The secondary film-forming agent may further comprise isopropyl alcohol. One preferred available product is sold under the trademark DERMACRYL polymer. One specific grade of DERMACRYL polymer is 79 (28-4979), sold by the National Starch & Chemical Company of Bridgewater, N.J.

Additional additives include spherical silica, which is readily available from a variety of sources. One suitable supply of silica spheres is sold under the trade name SPHERON by Presperse, Inc of Somerset, New Jersey. Another preferred additive is polytetrafluoroethylene (PTFE). One suitable product is sold under the trademark MICROSLIP by Chesham Chemicals Ltd. of Harrow, England. Preservatives include methyl parabin, propyl parabin and citric acid. The cumulative amount of the preservative(s) preferably is less than 0.5 wt. %. The cumulative amount of the secondary film-forming agent, silica and PTFE additives preferably is about 0.5 wt. % or slightly higher.

The following examples are intended to be illustrative and not limiting of the disclosed compositions.

### EXAMPLE 1

| Component | wt. % |
|---|---|
| trimethylsiloxysilicate (TMS) | 22.74 |
| hexamethyldisiloxane (dimethicone) | 46.34 |
| color paste* | 30.00 |
| DERMACRYL 79 polymer solution (1 | .50 |
| part by weight carboxylated acrylic | |
| copolymer in 15 parts by weight 70% | |
| isopropyl alcohol) | |
| SPHERON (silica) | 0.02 |
| MICROSLIP (PTFE) | 0.02 |
| methyl paraben | 0.2 |
| propyl paraben | 0.1 |
| butyl paraben | 0.05 |
| citric acid | 0.03 |
| | 100.00 |

*Color Paste: (1) pigment, isopropyl titanium triisostearate and (2) TMS and low viscosity dimethicones (polydimethylsiloxanes), wherein the TMS and dimethicone mixture has a viscosity of about 300 cps in a ratio of (1):(2) of 1:1 to 1:4.

Preferably, the TMS and the dimethicone solvent are mixed together until a clear, homogeneous mixture is obtained. Then, pre-mixed color paste is added. Next, the DERMACRYL polymer solution and PTFE are added and mixed until well dispersed prior to the addition of the various preservatives. The container holding the composition is sealed to prevent evaporation.

### EXAMPLE 2

| Component | wt. % |
|---|---|
| trimethylsiloxysilicate (TMS) | 23.8 |
| hexamethyldisiloxane (DM 0.65) | 45.56 |
| color paste* | 10 |
| DERMACRYL 79 polymer solution (1 | .24 |
| part by weight carboxylated acrylic | |
| copolymer in 15 parts by weight 70% | |
| isopropyl alcohol) | |
| SPHERON (silica) | 0.02 |
| MICROSLIP (PTFE) | 0.02 |
| Cloisonne Nu Antique Bronze | 10 |
| (pearlescent colorant) | |
| Cloisonne Nu Antique Rouge | 10 |
| (pearlescent colorant) | |
| methyl paraben | 0.2 |
| propyl paraben | 0.1 |
| butyl paraben | 0.04 |
| citric acid | 0.02 |
| | 100.00 |

*Color Paste: (1) pigment (iron oxide), isopropyl titanium triisostearate and (2) TMS and low viscosity dimethicones (polydimethylsiloxanes), wherein the TMS and dimethicone mixture has a viscosity of about 300 cps in a ratio of(1):(2) of 1:1 to 1:4.

Preferably, the TMS and the dimethicone solvent are mixed together until a clear, homogeneous mixture is obtained. Then, pre-mixed color paste is added. Next, the pearlescent pigments are added and mixed until dispersed well. Finally, the the DERMACRYL polymer solution, silica, and PTFE are added and mixed until well dispersed prior to the addition of the various preservatives. The container holding the composition is sealed to prevent evaporation.

FIGS. 1-9 provide illustration of an applicator 10 that can be used with the disclosed compositions. Because of the low viscosity of the dimethicones utilized, the resultant composition has a low viscosity and can be used with the felt tip applicator 10 of FIGS. 1-9.

Turning to FIG. 1, the applicator 10 includes a barrel 11 (see also FIG. 9) which includes an open end 12 having a threaded portion 13 that can be threadably connected to a corresponding threaded portion 14 of a cap 15 (see FIG. 3). The open end 12 of the barrel 11 also receives the cartridge casing 16 (FIG. 8) which, in turn, frictionally receives and engages a nib holder 17 (FIG. 5). The nib holder 17 and cartridge casing 16 combine to trap a mixing bar 18 and a mixing ball 19 in place, as shown in FIGS. 1 and 2. The open end 21 of the nib holder 17 receives and engages a nib or felt tip 22 (FIG. 4). The tip or nib 22 is felt or made of fibrous material, and can be selected from those commonly used in writing instruments. Because of the low viscosity of the disclosed eyeliner and lip liner compositions, a felt tip 22 can be utilized. The mixing bar 18 and mixing ball 19 help regulate flow from the cartridge 16 through the nib holder 17 to the nib 22.

While only certain embodiments have been set forth and described, alternative embodiments and various modifications will be apparent from the above descriptions to those skilled in the art. These and other alternatives are considered equivalents and within the scope of this disclosure.

Throughout the specification, where compositions are described as including components or materials, it is contemplated that the compositions can also consist essentially of, or consist of, any combination of the recited components or materials, unless described otherwise.

The practice of a method disclosed herein, and individual steps thereof, can be performed manually an/or with the aid of electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

What is claimed:

1. An extended wear cosmetic composition comprising:
a silicone resin;
a volatile dimethicone solvent;
a dimethicone dispersant having a viscosity of less than 1000 cps; and
a colorant.

2. The composition of claim 1, wherein the dimethicone dispersant has a viscosity ranging from about 5 cps to about 500 cps.

3. The composition of claim 1, wherein the at least one dimethicone dispersant has a viscosity of about 200 cps.

4. The composition of claim 1, further comprising a secondary film-forming agent.

5. The composition of claim 4, wherein the secondary film-forming agent comprises a hydrophobic acrylic copolymer.

6. The composition of claim 5, wherein the hydrophobic acrylic copolymer is carboxylated and has a molecular weight in excess of 10,000 Da.

7. The composition of claim 1, having a viscosity of about 1000 cps or less.

8. The composition of claim 1, wherein the silicone resin comprises trimethylsiloxysilicate.

9. The composition of claim 1, wherein the dimethicone solvent comprises hexamethyldisiloxane.

10. The composition of claim 1, wherein the at least one dimethicone dispersant comprises polydimethylsiloxane.

11. An extended wear cosmetic composition comprising:
from about 10 wt. % to about 30 wt. % of a silicone resin;
from about 35 wt. % to about 55 wt. % of a volatile dimethicone solvent; and
greater than 20 wt. % of a color paste; the color paste comprising a dimethicone dispersant having a viscosity of less than 1,000 cps.

12. The composition of claim 11, wherein the dimethicone dispersant has a viscosity ranging from about 5 cps to about 500 cps.

13. The composition of claim 11, wherein the dimethicone dispersant has a viscosity of about 200 cps.

14. The composition of claim 11, further comprising a secondary film-forming agent.

15. The composition of claim 14, wherein the secondary film-forming agent comprises a hydrophobic acrylic copolymer.

16. The composition of claim 15, wherein the hydrophobic acrylic copolymer is carboxylated and has a molecular weight in excess of 10,000 Da.

17. The composition of claim 11, having a viscosity of about 1000 cps or less.

18. The composition of claim 11, wherein the silicone resin comprises trimethylsiloxysilicate.

19. The composition of claim 11, wherein the dimethicone solvent comprises hexamethyldisiloxane.

20. The composition of claim 11, wherein the dimethicone dispersant comprises polydimethylsiloxane.

21. An extended wear cosmetic composition comprising:
from about 10 wt. % to about 30 wt. % of trimethylsiloxysilicate;
from about 35 wt. % to about 55 wt. % of hexamethyldisiloxane;
greater than 25 wt. % of a color paste comprising about 40 wt. % colorant and about 60 wt. % of a mixture of a silicone resin and at least two polydimethylsiloxanes, the polydimethylsiloxanes having a combined viscosity of less than 500 cps; and
from about 0.25 wt. % to about 1 wt. % of a secondary film-forming agent.

22. The composition of claim 21, wherein the at least two polydimethylsiloxanes have a combined viscosity ranging from about 150 cps to about 350 cps.

23. The composition of claim 21, wherein the at least two polydimethylsiloxanes have a combined viscosity of less than 300 cps.

24. The composition of claim 21, wherein the secondary film-forming agent comprises a high molecular weight hydrophobic carboxylated acrylic copolymer.

25. The composition of claim 21, wherein the color paste comprises an isopropyl titanium triisostearate-treated pigment.

26. A method for making an extended wear cosmetic composition, the method comprising:
mixing a silicone resin with a volatile dimethicone solvent;
blending a colorant with additional silicone resin and a dimethicone dispersant having a viscosity of less than 1,000 cps to form a colorant blend;
adding the colorant blend to the mixed silicone resin and volatile dimethicone solvent;
adding a secondary film-forming agent;
adding a resin selected from the group consisting of silica resins, polytetrafluroethylene resins and mixtures thereof;
adding a preservative; and
mixing the resultant product.
